(19) United States
(12) Patent Application Publication
(10) Pub. No.: US 2019/0247223 A1
(43) Pub. Date: Aug. 15, 2019

(54) NON-INVASIVE URINE COLLECTION DEVICE

(71) Applicants: Francisco Brun, San Rafael, CA (US); Lindsey Gabrielson, San Rafael, CA (US)

(72) Inventors: Francisco Brun, San Rafael, CA (US); Lindsey Gabrielson, San Rafael, CA (US)

(21) Appl. No.: 16/396,221

(22) Filed: Apr. 26, 2019

Related U.S. Application Data
(63) Continuation-in-part of application No. PCT/US2017/059081, filed on Oct. 30, 2017.
(60) Provisional application No. 62/415,114, filed on Oct. 31, 2016.

(51) Int. Cl. A61F 5/455; A61F 5/44; A61F 5/453

(52) U.S. Cl. CPC A61F 5/4556; A61F 5/4405; A61F 5/4408; A61F 5/453; A61F 5/455

(57) ABSTRACT

A non-invasive urine collection device is disclosed that can be used by both male persons and female persons. For example, the urine collection device is designed such that through orientation and/or deformation, the urine collection device may be configured to seal around the human pelvic area to sanitarily and non-invasively collect urine. The collection device comprises a funnel with a drain as a first end and a receptacle opening at a second end opposite the first end, a handle on one side, and a flexible ring seal along the perimeter of the receptacle opening. The flexible ring seal, shape of the receptacle opening, and the angle of the funnel wall in relation to the receptacle opening and funnel drain is such that the collection device can be used in either a handle up or handle down orientation.

### CROSS-REFERENCE TO RELATED APPLICATION(S)

This application is a continuation-in-part of, and claims the benefit of, International Application No. PCT/US2017/059081, filed Oct. 30, 2017, which claimed priority to U.S. Provisional Application No. 62/415,114, filed Oct. 31, 2016, both of which are expressly and entirely incorporated herein by reference.

### FIELD OF THE INVENTION

The present invention relates to a flexible urine collection device for use with both male and female patients, and methods of use.

### BACKGROUND

Generally, in-dwelling catheters are often used to control urine disposal for people who cannot easily get out of bed (e.g., bed-bound hospital patients). However, such catheters are invasive, can be painful, and can cause urinary tract infections and bladder infections. Thus, the use of in-dwelling catheters with people who cannot easily get out of bed (or their wheelchair) but who are cognitively aware enough to otherwise manage their personal waste elimination, can lead to undue medical and comfort issues. Alternatives to in-dwelling catheters include condom catheters, and hard plastic urinals or bed pans, which can be difficult to manipulate and are prone to spillage. Furthermore one size or shape may not work for all patients nor accommodate different positions of the patient.

What is needed is a non-invasive urine collection device that can accommodate users of regardless of size, shape, gender, or position.

### SUMMARY

The present invention provides a non-invasive urine collection device that can be used by both male persons and female persons. For example, the urine collection device is designed such that through orientation and/or deformation, the urine collection device may be configured to seal around the human pelvic area to sanitarily and non-invasively collect urine. The collection device comprises a funnel with a drain as a first end and a receptacle opening at a second end opposite the first end, a handle on one side, and a flexible ring seal along the perimeter of the receptacle opening. The flexible ring seal, shape of the receptacle opening, and the angle of the funnel wall in relation to the receptacle opening and funnel drain is such that the collection device can be used in either a handle up or handle down orientation.

In accordance with example embodiments of the present invention, a non-invasive urine collection device is provided. The collection device includes a collection funnel having a shape configured to receive urine there through. The collection funnel includes a handle disposed on one side, a circumnavigating funnel wall, a drain at a first end of the funnel, a receptacle opening at a second end of the funnel opposite the first end, and a raised flexible ring seal disposed along the perimeter of the receptacle opening.

The funnel wall circumnavigates from a handle-side-wall portion around to a drain-side-wall portion that is opposite the handle-side-wall portion and back to the handle-side-wall.

The receptacle opening has, across a cross-sectional plane of the receptacle opening, a generally elliptical or oblong shape with a length to width ratio of about 5:4, wherein the length of the opening is measured between a handle-side-wall of the collection funnel and the drain-side-wall of the collection funnel.

The angle between the cross-sectional plane of the receptacle opening and a longitudinal axis of the handle-side-wall is between about 40 degrees and about 80 degrees and an angle between the cross-sectional plane of the receptacle opening and a longitudinal axis of the drain-side-wall is between about 100 degrees and about 130 degrees.

In accordance with an aspect of the invention, the collection funnel is configured to be flexed to alter a shape of the receptacle opening.

In accordance with another aspect of the invention, the collection device further comprises a tube connected to the collection funnel at a drain of the funnel, the tube configured to receive the collected urine.

In accordance with another aspect of the invention, the collection device is sized, dimensioned, configured, and structured to be operable handle-side-wall up and operable handle-side-wall down.

In accordance with another aspect of the invention, the collection device is sized, dimensioned, configured, and structured to collect urine from a male or female anatomy in a standing position, seated position, and/or a supine position by positioning the device handle-side-wall up or operable handle-side-wall down.

In accordance with another aspect of the invention, the angle between the cross-sectional plane of the receptacle opening and a longitudinal axis of the handle-side-wall is about 60 degrees.

In accordance with another aspect of the invention, the angle between the cross-sectional plane of the receptacle opening and a longitudinal axis of the drain-side-wall is 110 degrees.

In accordance with another aspect of the invention, the collection funnel is flexible.

In accordance with another aspect of the invention, the seal comprises a flexible raised ring configured to seal about the human pelvic area and configured to prevent urine from leaking out of the funnel opening.

In accordance with another aspect of the invention, the drain is configured to receive a tube for coupling with a collection bag or wall suction.

In accordance with another aspect of the invention, the collection device further comprises a backflow prevention mechanism disposed proximal the drain and configured to resist backflow of fluid from the drain toward the receptacle opening. In certain aspects, the backflow prevention mechanism comprises the drain having a wall flared at about 4.5 degrees relative to an interior wall of the device.

In another aspect, the present invention provides an ornamental design for a urine collection device, as illustrated in FIGS. 7-13.

Any of the features or embodiments discussed above or herein may be combined in alternative embodiments.

### BRIEF DESCRIPTION OF THE FIGURES

These and other characteristics of the present invention will be more fully understood by reference to the following detailed description in conjunction with the attached drawings, in which:

FIG. 1 is a side view of a urine collection device, in accordance with an example embodiment;

FIG. 2 is a front view of a urine collection device configured in a male collection mode, in accordance with an example embodiment;

FIG. 3 is a front view of a urine collection device configured in a female collection mode, in accordance with an example embodiment;

FIG. 4 is a side view of a urine collection device, in accordance with another example embodiment;

FIG. 5 is a front view of a urine collection device configured in a male collection mode, in accordance with another example embodiment;

FIG. 6 is a front view of a urine collection device configured in a female collection mode, in accordance with another example embodiment;

FIG. 7 is a rear perspective view of a urine collection device;

FIG. 8 is a front perspective view of a urine collection device;

FIG. 9 is a front view of a urine collection device;

FIG. 10 is a rear view of a urine collection device;

FIG. 11 is a top view of a urine collection device;

FIG. 12 is a side view of a urine collection device; the opposite side is identical; and

FIG. 13 is a bottom view of a urine collection device.

### DETAILED DESCRIPTION

An illustrative embodiment of the present invention relates to a non-invasive urine collection device that can be used by both male persons and female persons. For example, the urine collection device is designed such that through orientation and/or deformation, the urine collection device may be configured to seal around the human pelvic area to sanitarily and non-invasively collect urine. The collection device comprises a funnel with a drain as a first end and a receptacle opening at a second end opposite the first end, a handle on one side, and a flexible ring seal along the perimeter of the receptacle opening. The flexible ring seal, shape of the receptacle opening, and the angle of the funnel wall in relation to the receptacle opening and funnel drain is such that the collection device can be used in either a handle up or handle down orientation.

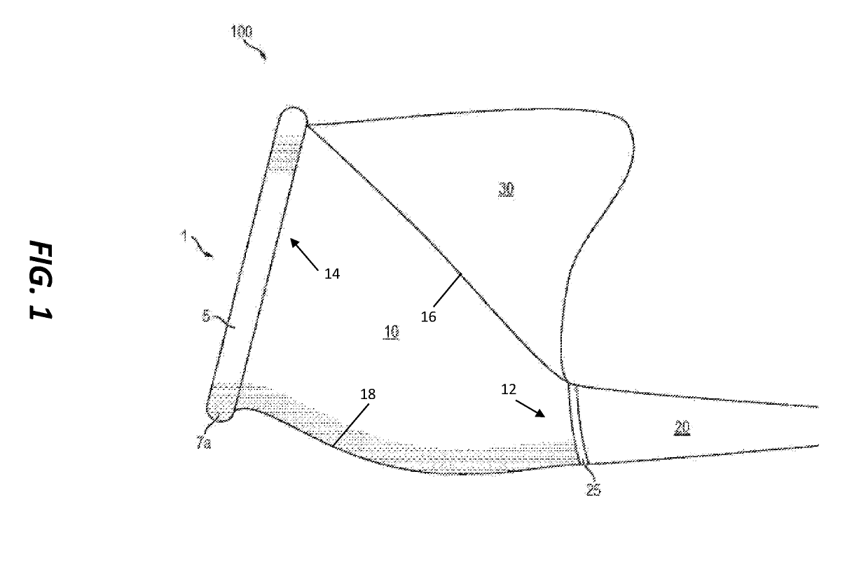

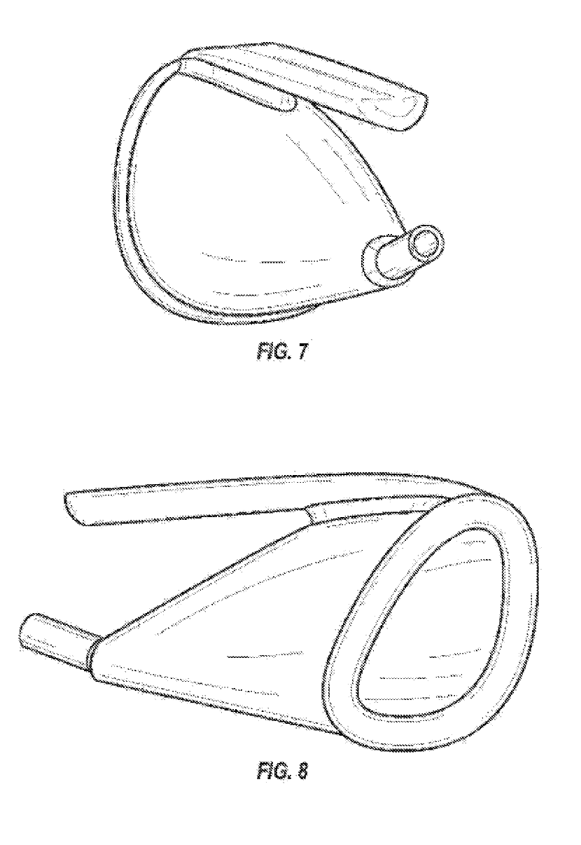

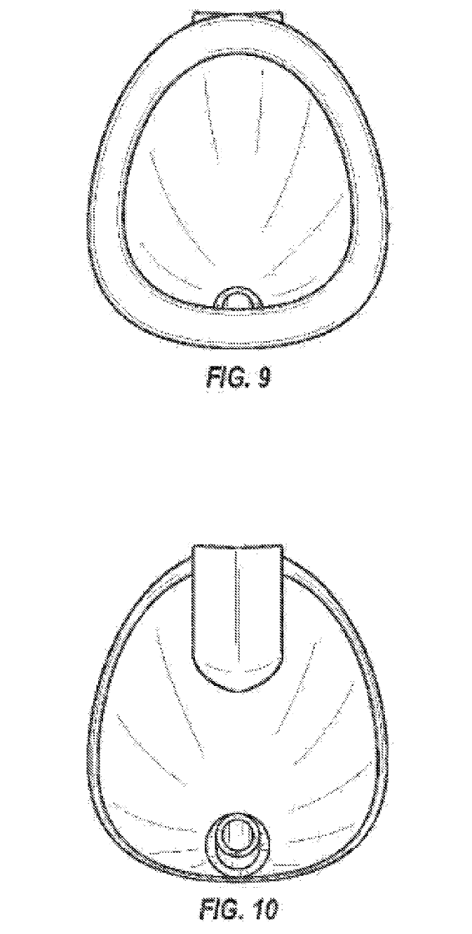

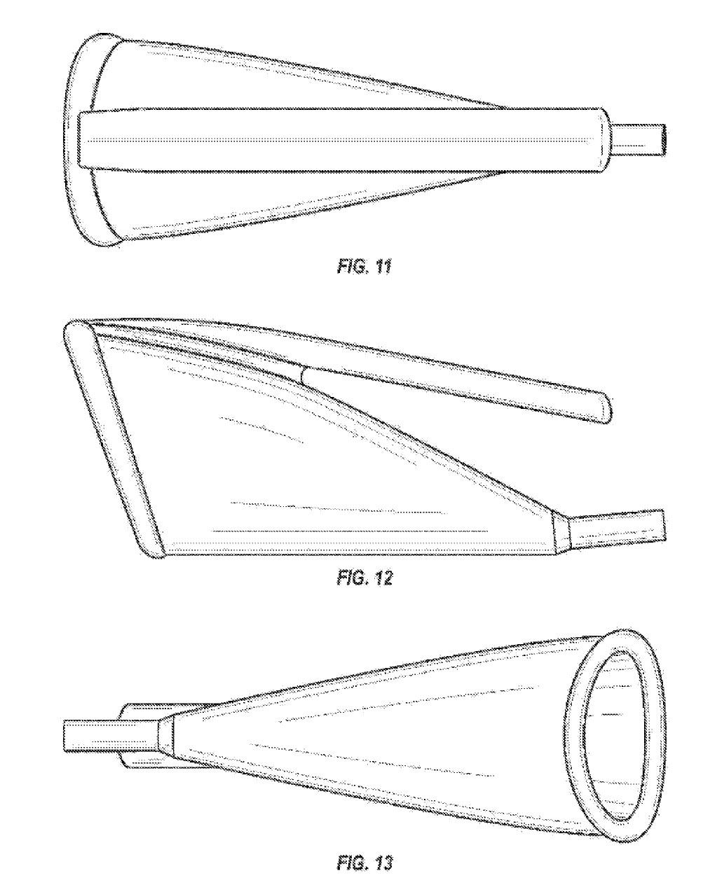

FIGS. 1 through 13 wherein like parts are designated by like reference numerals throughout, illustrate an example embodiment or embodiments of a non-invasive urine collection device, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

The present invention now will be described more fully hereinafter with reference to the accompanying drawings, in which some, but not all embodiments of the invention are shown. Indeed, the invention may be embodied in many different forms and should not be construed as limited to the embodiments set forth herein Like numbers refer to like elements throughout.

FIGS. 1 through 6 illustrate example embodiments of a non-invasive urine collection device 100. In example embodiments, the urine collection device 100 comprises a collection funnel 10 having a drain 20 at a first end 12 of the collection funnel 10. In example embodiments, the collection funnel 10 comprises a receptacle opening 1 at a second end 14 of the collection funnel 10 and configured for receiving urine there through. The collection funnel 10 is formed by a circumnavigating funnel wall extending between the receptacle opening 1 and drain 20. The collection funnel 10 formed by the funnel wall is shaped and configured to direct the collected urine received at the receptacle opening into the drain 20. The drain 20 is configured to provide the collected urine to an appropriate disposal facility (e.g., to a urine storage/disposal device, to a suction device, and/or the like). In some cases, the drain 20 directs urine to a collection bag (optionally vented). In some cases, flexible tubing is connected to the drain 20 to direct urine or other fluid to the collection bag. In certain embodiments the drain 20 is shaped or otherwise configured for mating with tubing or other collection device. Accordingly, the drain 20 can be tubular in shape. In certain embodiments, the tube shape of the drain 20 can be flared to improve the securing of a tube or other fluid collection devices.

In example embodiments, a backflow prevention mechanism 25 is disposed at or near the junction of the collection funnel 10 and the drain 20. For example, the backflow prevention mechanism 25 may be disposed in the first end 12 of the collection funnel 10, within the drain 20, and/or between the first end 12 of the collection funnel 10 and the drain 20. In example embodiments, the backflow prevention mechanism 25 is a one-way valve, a back-wash valve, and/or the like configured to permit urine or other fluid to flow from the collection funnel 10 into the drain 20, and to prevent urine or other fluid from crossing from the drain 20 into the collection funnel 10. In some cases, the drain 20 is a flat tube that impedes backflow of the urine or other fluid. In other cases, the backflow prevention mechanism comprises a wall that is offset from the plane of the funnel to impede the flow of urine back through the funnel 10 from the drain 20 (e.g., by flaring the end as described below). In example embodiments, the urine collection device 100 further comprises a handle 30 located on a side of the funnel 10 opposite from the side the drain 20 is located. In some cases, the handle 30 may be configured for aiding a person using and/or operating the urine collection device 100 to hold the urine collection device 100 against their pelvic area. In some cases, the handle is an integrated part (e.g., a fin or blade) of the collection device.

Example embodiments of the collection funnel 10 comprise a raised flexible ring 5. The raised flexible ring 5 may be configured to seal the receptacle opening 1 about the user's pelvic area. In some cases, the raised flexible ring 5 may be inflatable (an inflated member), and for example, when the receptacle opening 1 is positioned about a user's pelvic area, the inflated member may be inflated to seal the receptacle opening 1 about the user's pelvic area. In some embodiments, the raised flexible ring 5 forms a ring around the circumference of the opening 1 of the collection funnel 10 to be placed against an individual's pelvic area. In some cases, the raised flexible ring 5 is comprised of an air-filled chamber or foam material that flexes when placed against a user's pelvic area to form a seal. For example, the raised flexible ring 5 may be configured to reduce and/or prevent leakage of urine from the receptacle opening 1 when the urine collection device 100 is in use to collect urine.

In example embodiments, the funnel wall of the collection funnel 10 and/or the raised ring 5 may be divided into a plurality of portions. For example, the collection funnel 10 and/or the raised flexible ring 5 may comprise a posterior portion 7a on a side 18 of the funnel 10 where the drain 20 located and an anterior portion 6a on a side 16 of the funnel 10 where the handle 30 is located. Additionally, the collection funnel 10 and/or the raised ring 5 may comprise first and second side portions 6b, 6c, and first and second anterior side portions 7b, 7c. In example embodiments, one or more portions of the funnel wall of collection funnel 10 and/or the raised flexible ring 5 may be flexible portions 6 (e.g., 6a, 6b, 6c) and one or more portions of the funnel wall of the collection funnel 10 and/or the raised flexible ring 5 may be firm portions 7 (e.g., 7a, 7b, 7c). For example, the posterior portion 7a, the first anterior side portion 7b, and/or the second anterior side portion 7c may be firm portions. For example, the anterior portion 6a, the first side portion 6b, and the second side portion 6c may be flexible portions. In example embodiments, the firm portions 7 (e.g., 7a, 7b, 7c) and the flexible portions 6 (e.g., 6a, 6b, 6c) may be configured to allow the urine collection device 100 to effectively seal about the user's pelvic area. For example, the posterior portion 7a may be a firm portion to aid the user in maintaining the urine collection device 100 sealed against the user's pelvic area. In some cases, the drain side wall 18 of the collection funnel 10 (opposite the handle side) is angled from the posterior portion 7a of the receptacle opening 1 to the drain 20 (or optional back flow valve 25) to cause urine or other fluid to flow from the receptacle opening 1 toward and into the drain 20. This configuration aids in preventing leakage when a user (e.g., a bed-bound patient) is in a lying position (e.g., supine). An example of this can be seen in FIGS. 4-6.

In the example of FIGS. 4-6, the funnel 10 has a handle 30 disposed on one side 16, a circumnavigating funnel wall, a drain 20 at a first end 12, and a receptacle opening 1 at a second end 14 opposite of the first end 12. The funnel being formed by the funnel wall circumnavigating from the handle-side-wall 16 portion around to the drain-side-wall 18 portion that is opposite the handle-side-wall portion and back to the handle-side-wall. The raised flexible ring 5 is disposed around the perimeter of the receptacle opening 1.

Referring now to FIG. 4, it can be seen that funnel 10 has the handle-side-wall 16 and the drain-side-wall 18 angled in relation to the receptacle opening 1 to direct urine flow toward the drain 20. The angle 40 between the cross sectional plane of receptacle opening 1 and the longitudinal axis of the handle-side-wall is between about forty degrees (40°) and about eighty degrees (80°). In certain embodiments, the angle 40 between the cross sectional plane of the receptacle opening 1 and the longitudinal axis of the handle-side-wall is about sixty degrees (60°). The angle 42 between the cross section plane of the receptacle opening 1 and the longitudinal axis of the drain-side-wall 18 is between about one hundred degrees (100°) and about one hundred thirty degrees (130°). In certain embodiments, the angle 42 between the cross section plane of the receptacle opening 1 and the longitudinal axis of the drain-side-wall 18 is between about one hundred ten degrees. (110°). These angles 40, 42 further ensure that urine flows from the receptacle opening 1 towards the drain 20 regardless of the collection device 100 being orientated on the user with the handle 30 on the top or on the bottom during use.

In this example, the backflow prevention mechanism 25 comprises a wall flared at about four and a half degrees (4.5°) relative to the longitudinal plane of the drain-side-wall 18.

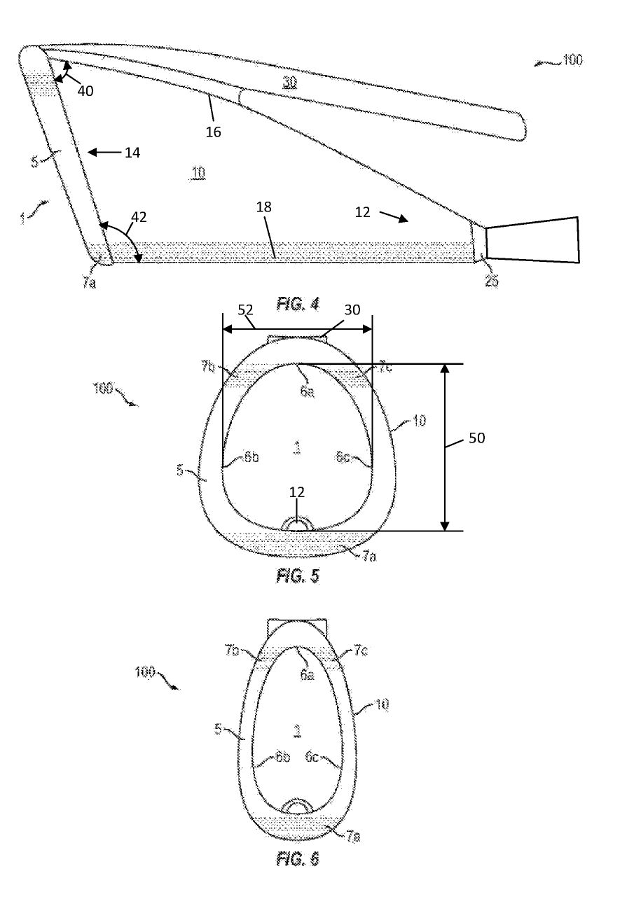

Referring now to FIG. 5, it can be seen that the receptacle opening 1 has a generally elliptical or oblong shape in an un-flexed or non-deformed state with a length to width ratio of about 5:4. The length 50 of the receptacle opening is measured between the handle-side-wall 16, here indicated by anterior portion 6a of the receptacle opening 1, and the drain side wall 18, here indicated by the posterior portion 7a of the receptacle opening 1. The width 52 is measure between the first side portion 6b and second side portion 6c of the funnel wall.

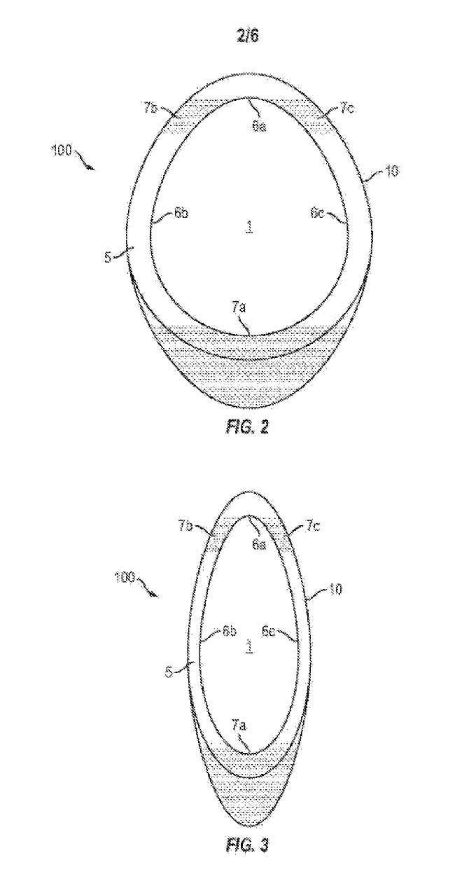

In example embodiments, the firm portions 7 (e.g., 7a, 7b, 7c) and the flexible portions 6 (e.g., 6a, 6b, 6c) may be configured to allow the collection funnel 10 to change shape so as to change the shape of the receptacle opening 1 defined thereby. For example, the shape of the opening 1 may be configurable based on flexing the funnel wall of the collection funnel 10 and/or the raised flexible ring 5. For example, the collection funnel 10 and/or the raised flexible ring 5 may be re-shaped, thereby re-shaping the receptacle opening 1. For example, the shape of the funnel 10 (and the opening 1 and the raised flexible ring 5) may be changed to accommodate the pelvic area, urethra configuration and/or the like of the specific user from which urine is to be collected. For example, the collection funnel 10 (and the receptacle opening 1 and the raised flexible ring 5) may be switched, flexed, and/or the like between a male collection mode (shown in FIGS. 2 and 5) for collecting urine from a male user, and a female collection mode (shown in FIGS. 3 and 6) for collecting urine from a female user. In example embodiments, the shape of the receptacle opening 1 may be rounder in the male collection mode than in the female collection mode. In example embodiments, the shape of the funnel opening 1 may be more elongated, elliptical, and/or oblong in the female collection mode than in the male collection mode. In example embodiments, the collection funnel 10 (and the funnel opening 1 and the raised ring 5) in the male collection mode or in the female collection mode may be adjusted therefrom to provide a better fit (e.g., a better seal) about the user's pelvic area. For example, the collection funnel 10 may be configured to smoothly flex between the male collection mode and the female collection mode such that the receptacle opening 1 is shaped to provide a personalized seal about the user's pelvic area. In one embodiment, the collection funnel 10 is originally configured in the male collection mode and may be re-configured into the female collection mode by pressing together the outer edges of side portions 6b and 6c, release of which will cause the collection funnel 10 to resume the original shape associated with the male collection mode.

In other embodiments, the collection funnel 10 may be switched from male collection mode to female collection mode by inverting the orientation of the funnel opening 1 such that the anterior portion 6a, or handle side, and posterior portion 7a, or drain side, of the receptacle opening 1 switch positions in relation to the user's pelvic area. In still other embodiments, the collection funnel may be oriented however it best makes a seal to user's pelvic area regardless of user's gender. Furthermore, different orientations or configurations may work better if the user is seated versus supine or laying down. The ideal orientation and or configuration may also depend on the gender or the user who is sitting, supine or laying down. For example, for male users, a handle up orientation typically works best to establish a seal for the device regardless of whether the user is sitting or lying down. However, for a female user, a handle up orientation typically works better to establish a seal with the device if the user is supine or laying down. If the female user is sitting up, a handle down orientation may work better to establish a seal with the device.

In various embodiments, the funnel wall forming collection funnel 10 can be made from flexible polymer materials and foams. The wall can be between 1-3 mm in thickness. The materials can be non-absorbent or comprise an outer surface that is non-absorbent. In some cases, the urine collection device 100 is comprised of a single integrated piece composed of a uniform material throughout, optionally excepting the firm portions 7 (e.g., 7a, 7b, 7c), which can be comprised of a denser form of the same material.

In use, the non-invasive urine collection device is (i) configured into a male or female collection mode, (ii) placed against the user's pelvic area while the user evacuates his or her bladder, and (iii) removed from the user's pelvic area. In some cases, a caregiver (e.g., hospital staff) may configure the device, place the device against a patient's pelvic area while the patient evacuates his or her bladder, and then remove the urine collection device. In some embodiments, in which the raised ring is an inflated member (e.g., an air-filled chamber), the inflated member can be inflated prior to placing the collection device against the user's pelvic area. In some embodiments, the urine collection device can be used for an individual in a seated position. In some cases, the dimensions of the urine collection device can be scaled to accommodate smaller individuals (e.g., children).

Many modifications and other embodiments of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation, unless otherwise noted.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A non-invasive urine collection device, comprising:
a collection funnel having a shape configured to receive urine there through, the collection funnel comprising:
a funnel with a handle disposed on one side, the funnel having a circumnavigating funnel wall, a drain at a first end of the funnel, and a receptacle opening at a second end of the funnel opposite the first end, wherein the funnel wall circumnavigates from a handle-side-wall portion around to a drain-side-wall portion that is opposite the handle-side-wall portion and back to the handle-side-wall;
the receptacle opening having, across a cross-sectional plane of the receptacle opening, a generally elliptical or oblong shape with a length to width ratio of about 5:4, wherein the length of the opening is measured between a handle-side-wall of the collection funnel and the drain-side-wall of the collection funnel; and
a raised flexible ring seal disposed along a perimeter of the receptacle opening;
wherein an angle between the cross-sectional plane of the receptacle opening and a longitudinal axis of the handle-side-wall is between about 40 degrees and about 80 degrees and an angle between the cross-sectional plane of the receptacle opening and a longitudinal axis of the drain-side-wall is between about 100 degrees and about 130 degrees.

2. The device of claim 1, wherein the collection funnel is configured to be flexed to alter a shape of the receptacle opening.

3. The device of claim 1, further comprising a tube connected to the collection funnel at a drain in the funnel, the tube configured to receive the collected urine.

4. The device of claim 1, wherein the device is sized, dimensioned, configured, and structured to be operable handle-side-wall up and operable handle-side-wall down.

5. The device of claim 1, wherein the device is sized, dimensioned, configured, and structured to collect urine from a male or female anatomy in a standing position, seated position, and/or a supine position by positioning the device handle-side-wall up or operable handle-side-wall down.

6. The device of claim 1, wherein the angle between the cross-sectional plane of the receptacle opening and a longitudinal axis of the handle-side-wall is about 60 degrees.

7. The device of claim 1, wherein the angle between the cross-sectional plane of the receptacle opening and a longitudinal axis of the drain-side-wall is 110 degrees.

8. The device of claim 1, wherein the collection funnel is flexible.

9. The device of claim 1, wherein the seal comprises a flexible raised ring configured to seal about the human pelvic area and configured to prevent urine from leaking out of the funnel opening.

10. The device of claim 1, wherein the drain is configured to receive a tube for coupling with a collection bag or wall suction.

11. The device of claim 1, further comprising a backflow prevention mechanism disposed proximal the drain and configured to resist backflow of fluid from the drain toward the receptacle opening.

12. The device of claim 11, wherein the backflow prevention mechanism comprises the drain having a wall flared at about 4.5 degrees relative to an interior wall of the device.
